(19) United States
(12) Patent Application Publication
(10) Pub. No.: US 2026/0199486 A1
(43) Pub. Date: Jul. 16, 2026

(54) LATANOPROST ACID AND BRIMONIDINE CONJUGATES

(71) Applicant: DuNing Incorporated, Tustin, CA (US)

(72) Inventors: Shih-Horng Su, Irvine, CA (US); Hong-Jia Lin, Tainan (TW); Ching-Yi Chen, Tainan (TW); Wen-Chung Wu, Tainan (TW); Tina Tzee Ling Wong, Singapore (SG)

(21) Appl. No.: 19/263,127

(22) Filed: Jul. 8, 2025

Related U.S. Application Data
(60) Provisional application No. 63/745,190, filed on Jan. 14, 2025.

(51) Int. Cl. A61K 47/55; A61K 31/498; A61P 27/06

(52) U.S. Cl. CPC A61K 47/55; A61K 31/498; A61P 27/06

(57) ABSTRACT

Disclosed are compounds of formula (I). Each variable in the formula is defined. Also disclosed are a method of preparing such a compound, a pharmaceutical composition containing the compound, and a method for treating glaucoma using the compound.

### CROSS-REFERENCE TO RELATED APPLICATION

This application claims the benefit of U.S. Provisional Patent Application Ser. No. 63/745,190, filed on Jan. 14, 2025. The content of the prior application is incorporated by reference herein in its entirety.

### BACKGROUND

Elevated intraocular pressure (IOP) can lead to glaucoma and eventually vision loss and blindness if left untreated.

Topical eye drops are the first line of treatment, including prostaglandins and alpha-2 adrenergic agonists such as latanoprost, travoprost, tafluprost, bimatoprost, unoprost, brimonidine, apraclonidine, guanabenz, methyldopa, and lofexidine.

These medicines are effective in decreasing the pressure inside the eye. Nevertheless, they can be removed quickly by the body after topical administration, making their efficacy temporary. Take latanoprost as an example. Its elimination from human plasma is fast with a half-life of only 17 minutes. Approximately 88%-98% of a given dose is found in urine. See Xalatan® Package Insert for the 2.5 mL fill, page 4 (NDA 20-597/S-044; Reference ID: 3100250). To compensate its rapid elimination, a large amount of latanoprost is formulated in an eye drop, increasing its safety risk.

There is a need to develop a safe, effective, and long-lasting medicines for treating elevated IOP.

### SUMMARY

The present invention is based on an unexpected discovery that certain compounds are effective and long-lasting in reducing the IOP and treating glaucoma.

Accordingly, one aspect of the invention relates to compounds of formula (I) as shown below,

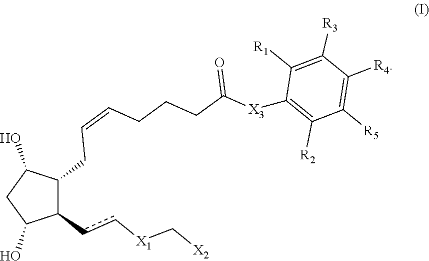

In formula (I) above, each of R₁, R₂, and R₅, independently, is H, halo, or OH; each of R₃ and R₄ is H, OH, or amino, or R₃ and R₄, together with the phenyl ring they are bonded to, form a quinoxaline ring; X₁ is CO, CHOH, or CF₂; X₂ is C₄-C₆ alkyl, C₄-C₆ alkyl-O—, aryl-O—, or aryl-CH₂—; X₃ is,

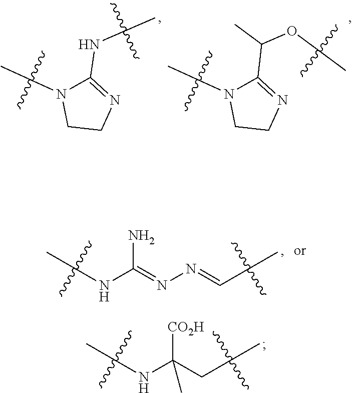

and [symbol] is a single or double bond.

Compounds of formula (I) can have one or any combination of the following preferred features:
(i) R₁ is H, Cl, or Br,
(ii) wherein R₂ is H or Cl,
(iii) R₅ is H or OH,
(iv) each of R₃ and R₄, independently, is H, OH, or NH₂,
(v) R₃ and R₄, together with the phenyl ring they are bonded to, form a quinoxaline ring, and the quinoxaline ring is unsubstituted or substituted with amino, hydroxyl, or halo,
(vi) X₁ is chiral CHOH,
(vii) X₂ is

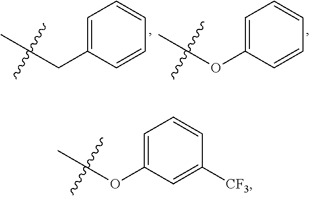

or n-hexyl,
(viii) X₃ is

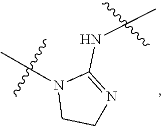 [SMILES: *NC1=NCCN1*]

and
(ix) [symbol] is a double bond.

A subset of the compounds of formula (I) includes the compounds of below formula (II):

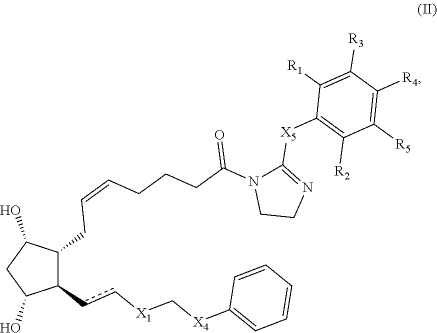

in which each of R₁, R₂, and R₅, independently, is H, F, Cl, or Br; each of R₃ and R₄ is H or NH₂, or R₃ and R₄, together with the phenyl ring they are bonded to, form a quinoxaline ring; X₁ is CHOH or CF₂; X₄ is CH₂ or O; and X₅ is NH or CH(CH₃)O.

Another aspect of this invention is a method of preparing the compound of claim 1, the method comprising the step of reacting a compound of formula (I-A) and a compound of formula (I-B):

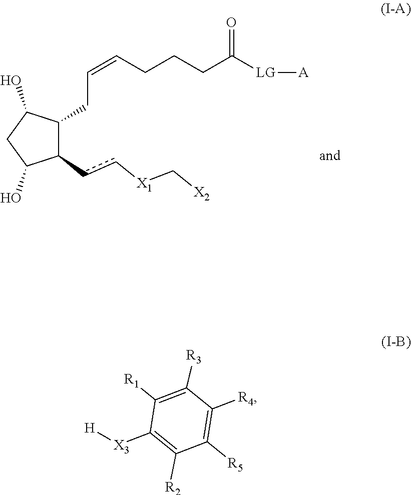

in which LG-A is a leaving group and X₃H is a nucleophile.

LG-A can be any suitable leaving group in a nucleophilic substitution reaction including halo,

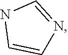

anhydride, N₃, or OH; and X₃H can be OH or amine.

Also within the scope of this invention are pharmaceutical compositions for treating IOP or glaucoma comprising any one of the compounds described above and a pharmaceutically acceptable salt.

Still within the scope of the invention is a method for treating glaucoma or elevated IOP comprising the step of administering to a subject in need thereof a compound or a pharmaceutical composition, both of which are described above. The invention also relates to use of such a compound (e.g., a pharmaceutical composition containing one of the compounds described above) for treating IOP or glaucoma or for the manufacture of a medicament for treating these conditions.

Unless defined otherwise, all technical and scientific terms used herein have the same meaning as is commonly understood by one of skill in art to which the subject matter herein belongs. As used herein, the following definitions are supplied in order to facilitate the understanding of the present invention.

Shown below are exemplary compounds of this invention, Compounds 1-25.

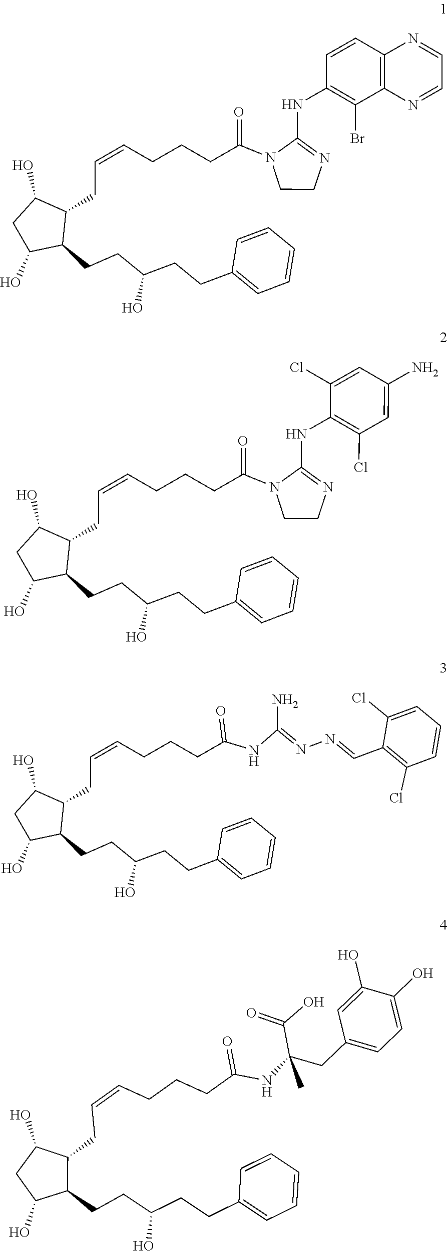 [SMILES: O=C(CCC/C=C\C[C@@H]1[C@@H](CC[C@@H](O)CCc2ccccc2)[C@H](O)C[C@@H]1O)N1CCN=C1Nc1ccc2nccnc2c1Br ; Nc1cc(Cl)c(NC2=NCCN2C(=O)CCC/C=C\C[C@@H]2[C@@H](CC[C@@H](O)CCc3ccccc3)[C@H](O)C[C@@H]2O)c(Cl)c1 ; N/C(=N\N=C\c1c(Cl)cccc1Cl)NC(=O)CCC/C=C\C[C@@H]1[C@@H](CC[C@@H](O)CCc2ccccc2)[C@H](O)C[C@@H]1O ; C[C@@](Cc1ccc(O)c(O)c1)(NC(=O)CCC/C=C\C[C@@H]1[C@@H](CC[C@@H](O)CCc2ccccc2)[C@H](O)C[C@@H]1O)C(=O)O]

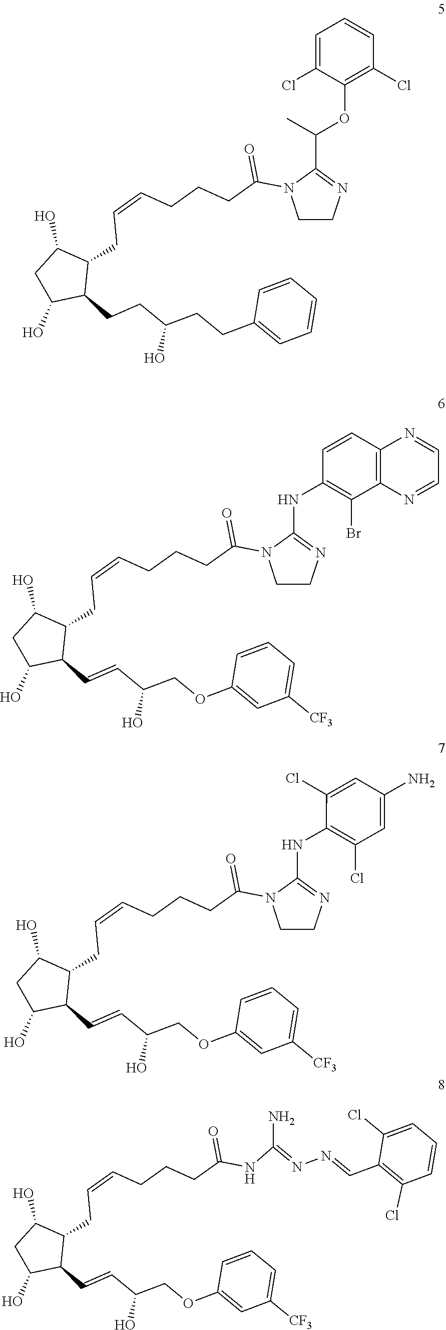 [SMILES: CC(Oc1c(Cl)cccc1Cl)C1=NCCN1C(=O)CCC/C=C\C[C@@H]1[C@@H](CC[C@@H](O)CCc2ccccc2)[C@H](O)C[C@@H]1O ; O=C(CCC/C=C\C[C@@H]1[C@@H](/C=C/[C@@H](O)COc2cccc(C(F)(F)F)c2)[C@H](O)C[C@@H]1O)N1CCN=C1Nc1ccc2nccnc2c1Br ; Nc1cc(Cl)c(NC2=NCCN2C(=O)CCC/C=C\C[C@@H]2[C@@H](/C=C/[C@@H](O)COc3cccc(C(F)(F)F)c3)[C@H](O)C[C@@H]2O)c(Cl)c1 ; N/C(=N\N=C\c1c(Cl)cccc1Cl)NC(=O)CCC/C=C\C[C@@H]1[C@@H](/C=C/[C@@H](O)COc2cccc(C(F)(F)F)c2)[C@H](O)C[C@@H]1O]

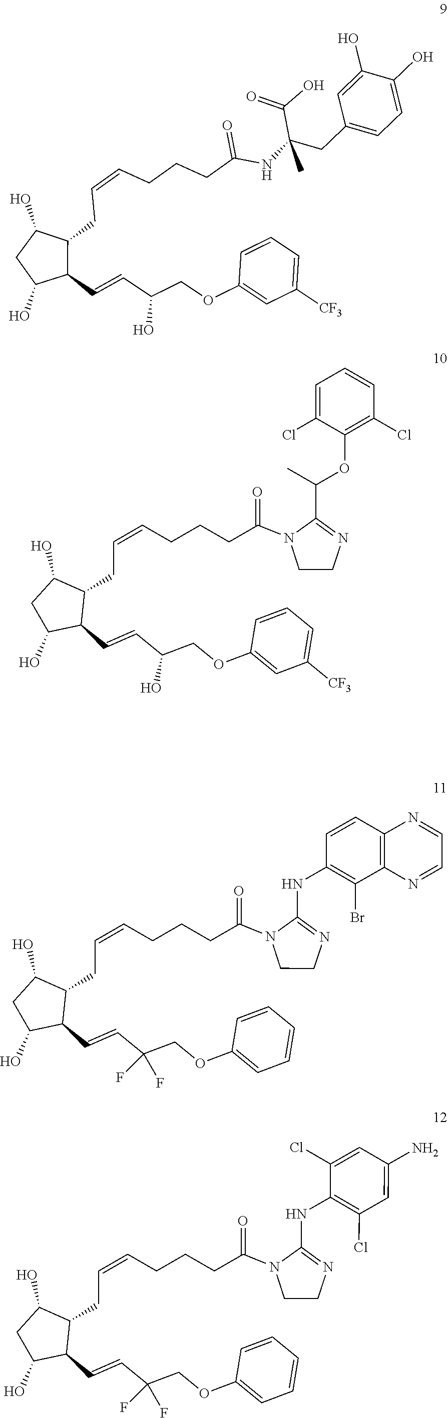 [SMILES: C[C@@](Cc1ccc(O)c(O)c1)(NC(=O)CCC/C=C\C[C@@H]1[C@@H](/C=C/[C@@H](O)COc2cccc(C(F)(F)F)c2)[C@H](O)C[C@@H]1O)C(=O)O ; CC(Oc1c(Cl)cccc1Cl)C1=NCCN1C(=O)CCC/C=C\C[C@@H]1[C@@H](/C=C/[C@@H](O)COc2cccc(C(F)(F)F)c2)[C@H](O)C[C@@H]1O ; O=C(CCC/C=C\C[C@@H]1[C@@H](/C=C/C(F)(F)COc2ccccc2)[C@H](O)C[C@@H]1O)N1CCN=C1Nc1ccc2nccnc2c1Br ; Nc1cc(Cl)c(NC2=NCCN2C(=O)CCC/C=C\C[C@@H]2[C@@H](/C=C/C(F)(F)COc3ccccc3)[C@H](O)C[C@@H]2O)c(Cl)c1]

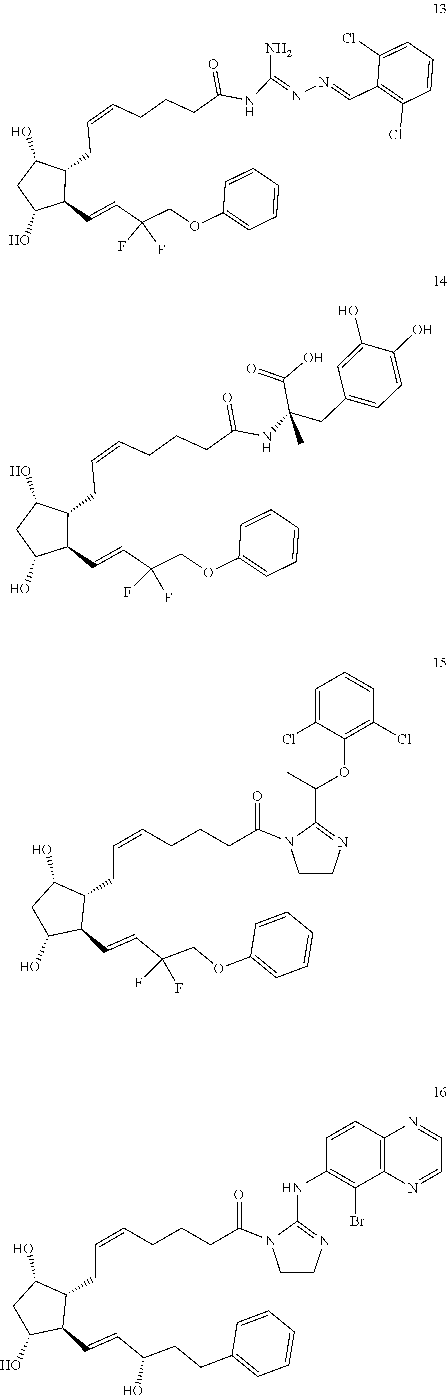 [SMILES: N/C(=N\N=C\c1c(Cl)cccc1Cl)NC(=O)CCC/C=C\C[C@@H]1[C@@H](/C=C/C(F)(F)COc2ccccc2)[C@H](O)C[C@@H]1O ; C[C@@](Cc1ccc(O)c(O)c1)(NC(=O)CCC/C=C\C[C@@H]1[C@@H](/C=C/C(F)(F)COc2ccccc2)[C@H](O)C[C@@H]1O)C(=O)O ; CC(Oc1c(Cl)cccc1Cl)C1=NCCN1C(=O)CCC/C=C\C[C@@H]1[C@@H](/C=C/C(F)(F)COc2ccccc2)[C@H](O)C[C@@H]1O ; O=C(CCC/C=C\C[C@@H]1[C@@H](/C=C/[C@@H](O)CCc2ccccc2)[C@H](O)C[C@@H]1O)N1CCN=C1Nc1ccc2nccnc2c1Br]

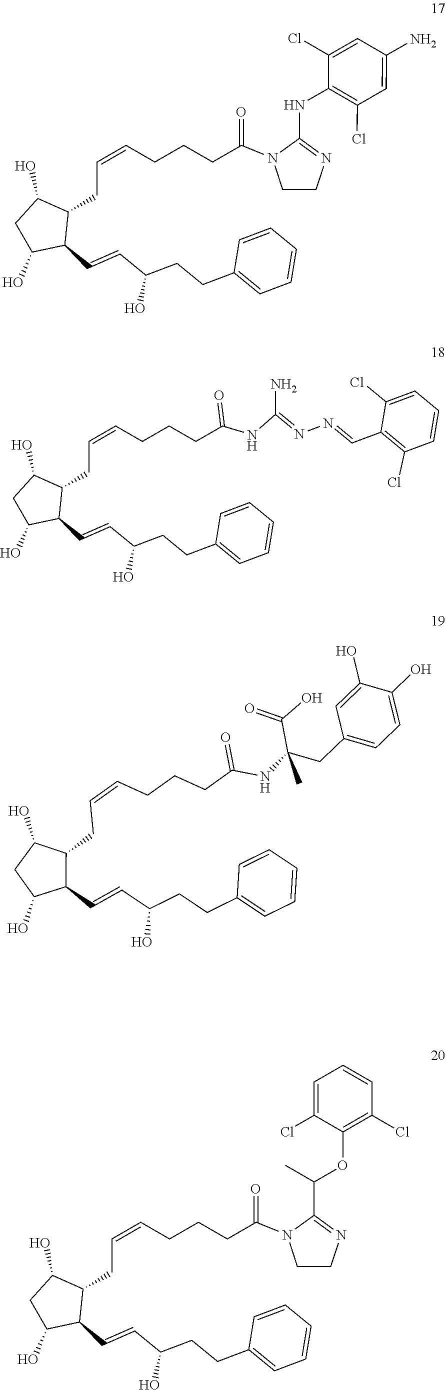 [SMILES: Nc1cc(Cl)c(NC2=NCCN2C(=O)CCC/C=C\C[C@@H]2[C@@H](/C=C/[C@@H](O)CCc3ccccc3)[C@H](O)C[C@@H]2O)c(Cl)c1 ; N/C(=N\N=C\c1c(Cl)cccc1Cl)NC(=O)CCC/C=C\C[C@@H]1[C@@H](/C=C/[C@@H](O)CCc2ccccc2)[C@H](O)C[C@@H]1O ; C[C@@](Cc1ccc(O)c(O)c1)(NC(=O)CCC/C=C\C[C@@H]1[C@@H](/C=C/[C@@H](O)CCc2ccccc2)[C@H](O)C[C@@H]1O)C(=O)O ; CC(Oc1c(Cl)cccc1Cl)C1=NCCN1C(=O)CCC/C=C\C[C@@H]1[C@@H](/C=C/[C@@H](O)CCc2ccccc2)[C@H](O)C[C@@H]1O]

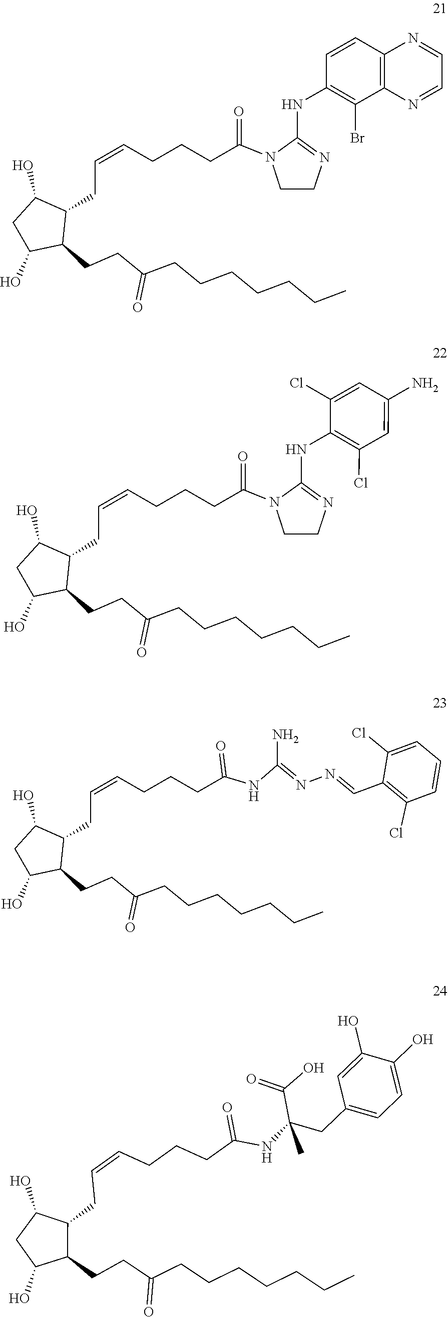 [SMILES: CCCCCCCC(=O)CC[C@@H]1[C@@H](C/C=C\CCCC(=O)N2CCN=C2Nc2ccc3nccnc3c2Br)[C@@H](O)C[C@H]1O ; CCCCCCCC(=O)CC[C@@H]1[C@@H](C/C=C\CCCC(=O)N2CCN=C2Nc2c(Cl)cc(N)cc2Cl)[C@@H](O)C[C@H]1O ; CCCCCCCC(=O)CC[C@@H]1[C@@H](C/C=C\CCCC(=O)N/C(N)=N/N=C/c2c(Cl)cccc2Cl)[C@@H](O)C[C@H]1O ; CCCCCCCC(=O)CC[C@@H]1[C@@H](C/C=C\CCCC(=O)N[C@@](C)(Cc2ccc(O)c(O)c2)C(=O)O)[C@@H](O)C[C@H]1O]

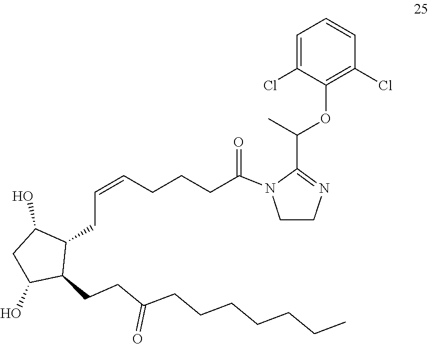 [SMILES: CCCCCCCC(=O)CC[C@@H]1[C@@H](C/C=C\CCCC(=O)N2CCN=C2C(C)Oc2c(Cl)cccc2Cl)[C@@H](O)C[C@H]1O]

The subject is a mammal such as a rodent, a canine, a primate, an equine, a high value agricultural animal, and a human, preferably a human.

The effective amount of the composition has the compound in a range from 0.01 ng to 10 mg per application.

The details of one or more embodiments of the invention are set forth in the drawings and description below. Other features, objects, and advantages of the invention will be apparent from the drawings and description and from the claims.

### BRIEF DESCRIPTION OF THE DRAWINGS

The foregoing and other objects, features and advantages will be apparent from the following description of particular embodiments of the disclosure, as illustrated in the accompanying drawings.

FIG. 1 shows the NMR spectrum of Compound 1.

FIG. 2A shows HPLC traces of samples of Compound 1 (LBJ) incubated with 1 mM HCl for the number of days indicated on each trace. LPA=latanoprost acid, BM=brimonidine.

FIG. 2B shows HPLC traces of samples of LBJ incubated with 1 mM NaOH for the number of days indicated on each trace.

FIG. 3A shows HPLC traces of samples of Latanoprost (LP) incubated with 1 mM HCl for the number of days indicated on each trace.

FIG. 3B shows HPLC traces of samples of LP incubated with 1 mM NaOH for the number of days indicated on each trace.

FIG. 4 shows plots of normalized peak areas of LP and LBJ from the HPLC traces shown in FIGS. 2A, 2B, 3A, and 3B versus days of hydrolysis in HCl and NaOH.

FIG. 5 is a plot of percent reduction in intraocular pressure (IOP) versus days of treatment with the indicated drugs in rabbit eyes.

### DETAILED DESCRIPTION

Certain terminology is used in the following description for convenience only and is not limiting.

The term “halo” refers to H, F, Cl, Br, or I.

The term “alkyl” or “alkylene’ as used herein, means a straight or branched chain, monovalent or divalent hydrocarbon. An alkyl group herein may have from 1 to 30 carbon atoms (e.g., 1-25, 2-20, 3-16, 5-8, 1-6, and 1-4) unless otherwise specified. An alkyl group may have 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, or a number of carbon atoms in a range from a first of the foregoing values to a second of the foregoing values, where the first and second values selected are any two of the foregoing values and the first value is less than the second. Examples include methyl (Me), ethyl, n-propyl, i-propyl, n-butyl, i-butyl, and t-butyl.

The term “alkoxy” refers to O-alkyl.

The term “aryl” herein refers to a monocyclic, bicyclic or tricyclic aromatic ring system. Examples include phenyl, biphenyl, 1- or 2-naphthyl, 1,2-dihydronaphthyl, 1,2,3,4-tetrahydronaphthyl, indenyl, and indanyl. Aryl can be unsubstituted or substituted with alkyl, alkenyl, alkynyl, cycloalkyl, heterocycloalkyl, aryl, heteroaryl, amino, ether, ester, and the like. The term “aralkyl” refers to alkyl substituted with aryl, i.e., aryl-alkyl.

The term “amino” refers to primary (NH₂), secondary (—NH—), tertiary

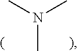

or quaternary

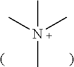

amine group bonding to or being included in one or more of C₁-C₃₀ (e.g., C₂-C₂₀ and C₄-C₁₆) aliphatic, C₁-C₃₀ (e.g., C₂-C₂₀ and C₄-C₁₆) heteroaliphatic, aryl, or heteroaryl moieties. Examples include alkyl amino, dialkyl amino, alkenyl amino, etc. Aliphatic amino examples include C₁-C₃₀ alkyl amino, C₂-C₃₀ alkenyl amino, C₂-C₃₀ alkynyl amino, and C₃-C₃₀ cycloalkyl. C₁-C₃₀ heterocycloalkyl amino is an example of heteroaliphatic amino.

Alkyl, alkylene, alkoxy, aryl, aralkyl, and amino mentioned herein include both substituted and unsubstituted moieties, unless specified otherwise. Examples of a substituent include deuterium (D), hydroxyl (OH), halo (e.g., F and Cl), amino (NH₂), cyano (CN), nitro (NO₂), alkyl, alkenyl, alkynyl, alkoxy, cycloalkyl, acylamino, alkylamino, aminoalkyl, haloalkyl (e.g., trifluoromethyl), heterocyclyl, alkoxycarbonyl, amido, carboxy (COOH), alkanesulfonyl, alkylcarbonyl, alkenylcarbonyl, carbamido, carbamyl, carboxyl, thioureido, thiocyanato, sulfonamido, aryl, arylamino, aralkyl, and heteroaryl. All substitutes can be further substituted.

The term “nucleophile” refers to a chemical moiety having a reactive pair of electrons. Any molecule or ion with electrons available for donation to another molecule, e.g., a free pair of electrons or at least one pi bond, can be considered as a nucleophile. Examples of nucleophiles include uncharged molecules or moieties such as water, amines, thiols, mercaptans and alcohols, and charged moieties such as alkoxides, thiolates, carbanions, and a variety of organic and inorganic anions.

The term “compound”, when referring to a compound of this invention, also includes its salts, solvates, and prodrugs. The pharmaceutically acceptable salts include those listed in Handbook of Pharmaceutical Salts: Properties, Selection and Use, 2nd Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A salt can be formed between an anion and a positively charged group (e.g., amino) on a compound. Examples of a suitable anion include chloride, bromide, iodide, sulfate, nitrate, phosphate, citrate, methanesulfonate, trifluoroacetate, acetate, malate, tosylate, tartrate, fumarate, succinate, glutamate, glucuronate, lactate, glutarate, and maleate. A salt can also be formed between a cation and a negatively charged group. Examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereomeric mixture, a cis-isomer, or a trans-isomer. Compounds of such isomeric forms are within the scope of this invention. They can be present as a mixture or can be isolated using chiral synthesis or chiral separation technologies.

The term “treating” or “treatment” refers to administering one or more of the compounds to a subject with the purpose to confer a therapeutic effect, e.g., to slow, interrupt, arrest, control, or stop of the progression of an existing disorder and/or symptoms thereof, but does not necessarily indicate a total elimination of all symptoms.

The term “substitute” refers to the ability to change one functional group, or moiety, of a compound for another functional group or moiety, provided that the valency of all atoms on the parent structure is maintained. The substituted group is interchangeably referred herein as “substitution” or “substituent.” When more than one position in any given structure is substituted with more than one substituent selected from a specified group, the substituent may be either the same or different at every position.

Numerical values or ranges (preceded by “about” or without it) refer to the explicitly recited numbers, and the numbers within the experimental error of the measure contemplated. Embodiments described with the modifier “about” may be altered to remove “about” in order to form further embodiments herein. Likewise, embodiments described without the modifier “about” may be altered to add “about” in order to form further embodiments herein.

A range expressed as being between two numerical values, one as a low endpoint and the other as a high endpoint, includes the values between the numerical values and the low and high endpoints. Embodiments herein include subranges of a range herein, where the subrange includes a low and high endpoint of the subrange selected from any increment within the range selected from each single increment of the smallest significant figure, with the condition that the high endpoint of the subrange is higher than the low endpoint of the subrange.

Further embodiments herein include replacing one or more “including” or “comprising” in an embodiment with “consisting essentially of” or “consisting of” “Including” and “comprising,” as used herein, are open ended, include the elements recited, and do not exclude the addition of one or more other element. “Consisting essentially of” means that addition of one or more element compared to what is recited is within the scope, but the addition does not materially affect the basic and novel characteristics of the combination of explicitly recited elements. “Consisting of” refers to the recited elements, but excludes any element, step, or ingredient not specified.

The words “a” and “one,” as used in the claims and in the corresponding portions of the specification, are defined as including one or more of the referenced items unless specifically stated otherwise. This terminology includes the words above specifically mentioned, derivatives thereof, and words of similar import. The phrase “at least one” followed by a list of two or more items, such as “A, B, or C” or “A, B, and C” means any individual one of A, B or C as well as any combination thereof.

As used herein, the term “therapeutically effective amount” refers to the amount of a compound which is effective for producing a desired therapeutic effect. The therapeutic effect may be achieved at a reasonable benefit/risk ratio applicable to medical treatment. A “therapeutically effective amount” may refer to an amount sufficient to reduce IOP or alleviate glaucoma in any degree. A “therapeutically effective amount” may refer to an amount sufficient to cause a decrease in disease symptoms. A “therapeutically effective amount” may refer to an amount sufficient to cause a disappearance of disease symptoms.

Therapeutic efficacy may depend on effective amounts of active agents and time of administration necessary to achieve a desired result. Administering a composition may be a preventive measure. Administering of a composition may be a therapeutic measure to promote IOP reduction, to minimize complications associated with the slow development of glaucoma especially in patients prone to glaucoma or the elderly.

The exact dosage may be chosen by the clinician based on a variety of factors and in view of individual patients. Dosage and administration may be adjusted to provide sufficient levels of the active agent or agents or to maintain the desired effect. For example, factors which may be taken into account may include the type and severity of a disease; age and gender of the patient; drug combinations; and an individual response to therapy.

Therapeutic efficacy and toxicity of active pharmaceutical agents in a composition may be determined by standard pharmaceutical procedures, for example, by determining the therapeutically effective dose in 50% of the population (ED50) and the lethal dose to 50% of the population (LD50) in cells cultured in vitro or experimental animals. Compositions may be evaluated based on the dose ratio of toxic to therapeutic effects (LD50/ED50), called the therapeutic index, the large value of which may be used for assessment. The data obtained from cell and animal studies may be used in formulating a dosage for human use.

The therapeutically effective dose may be estimated initially from cell culture assays. A therapeutically effective dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

The amount of a compound administered will depend upon the particular therapeutic compound used, the disease or disorder being treated, the age, weight, and condition of the patient, and the judgment of the clinician. A therapeutically effective amount may be a dose from 0.001 ng to 50 mg of the compound per kilogram of body weight of the subject or per eye drop. A therapeutically effective amount may be a dose of 0.001 ng, 0.002 ng, 0.003 ng, 0.004 ng, 0.005 ng, 0.006 ng, 0.007 ng, 0.008 ng, 0.009 ng, 0.01 ng, 0.02 ng, 0.03 ng, 0.04 ng, 0.05 ng, 0.06 ng, 0.07 ng, 0.08 ng, 0.09 ng, 0.1 ng, 0.2 ng, 0.3 ng, 0.4 ng, 0.5 ng, 0.6 ng, 0.7 ng, 0.8 ng, 0.9 ng, 0.001 μg, 0.002 μg, 0.003 μg, 0.004 μg, 0.005 μg, 0.006 μg, 0.007 μg, 0.008 μg, 0.009 μg, 0.01 μg, 0.02 μg, 0.03 μg, 0.04 μg, 0.05 μg, 0.06 μg, 0.07 μg, 0.08 μg, 0.09 μg, 0.1 μg, 0.2 μg, 0.3 μg, 0.4 μg, 0.5 μg, 0.6 μg, 0.7 μg, 0.8 μg, 0.9 ag, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg of the compound in each application, or a value in a range from a first of the foregoing values to a second of the foregoing values, where the first and second values selected are any two of the foregoing values and the first value is less than the second. The composition may be a combination of different compounds used per treatment dose.

The terms “subject” means a human or animal. Preferably, the animal is a vertebrate such as a primate, rodent, domestic animal or game animal. Primates include chimpanzees, cynomolgus monkeys, spider monkeys, and macaques, e.g., Rhesus. The rodent may be selected from mice, rats, guinea pigs, woodchucks, ferrets, rabbits and hamsters. The domestic or game animals may be selected from cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or subject may be selected from the foregoing or a subset of the foregoing. A patient or subject may be selected from all of the above, but excluding one or more groups or species such as humans, primates or rodents. In an embodiment, the patient or subject may be a mammal, e.g., a primate, e.g., a human. The terms, “patient” and “subject” are used interchangeably herein. Preferably, the subject is a mammal. The mammal may be a human, non-human primate, mouse, rat, dog, cat, horse, cow, or swine but is not limited to these examples. Mammals other than humans may be subjects that represent animal models of a disease or disorder. In addition, the methods described herein may be directed to treating domesticated animals and/or pets. A subject may be male or female.

As used herein, the terms “administer,” “administering,” “administration,” or the like refer to the placement of a composition into a subject. The administration may be by a method or route which results in at least partial localization of the composition at a desired site. Placement at a desired site may lead to a production of a desired effect. A composition described herein may be administered by any appropriate route known in the art including, but not limited to, oral or parenteral routes, including intravenous, intramuscular, subcutaneous, transdermal, airway (aerosol), pulmonary, nasal, rectal, topical, buccal, or sublingual administration.

Exemplary modes of administration include, but are not limited to, injection, infusion, instillation, inhalation, or ingestion. “Injection” includes without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebral, and intrasternal injection and infusion. In an embodiment, the compositions may be administered by intravenous infusion or injection.

A compound of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition, Academic Press, 2020).

To practice the method of the present invention, a composition or a kit containing one or more of the above-described compounds can be administered alone or co-administered with at least one other pharmacologically active substance simultaneously, concurrently, sequentially, successively, alternately, or separately. Simultaneous administration, also referring to as concomitant administration, includes administration at substantially the same time. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

Methods for synthesizing the compounds of this invention are well known in the art. See, for example, R. Larock, Comprehensive Organic Transformations (2^{nd} Ed., VCH Publishers 1999); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); L. Paquette, ed., Encyclopedia of Reagents for Organic Synthesis (2^{nd} ed., John Wiley and Sons 2009); and N. J. O'Reilly, W. S. Derwin, H. C. Lin “Optically Pure (S)-6,7-Dimethoxy-1,2,3,4-Tetrahydro-3-Isoquinoline-carboxylic Acid and Asymmetric Hydrogenation Studies Related to Its Preparation”, Synthesis 7 (1990) 550-556.

The compounds of Formula (I) thus prepared can be initially screened using in vitro and in vivo assays for their potency in treating IOP or glaucoma in cells.

In addition to the compounds of formula (I) above, the compounds of formula (III) below are also envisioned and effective in treating IOP.

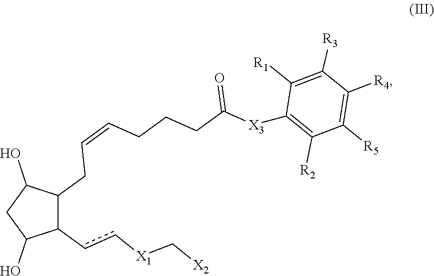

R₁ to R₅ and X₁ to X₃ are defined above.

The compounds of this invention can be prepared using a commercially available reagents and known compounds following methods such as the one depicted in Scheme I below.

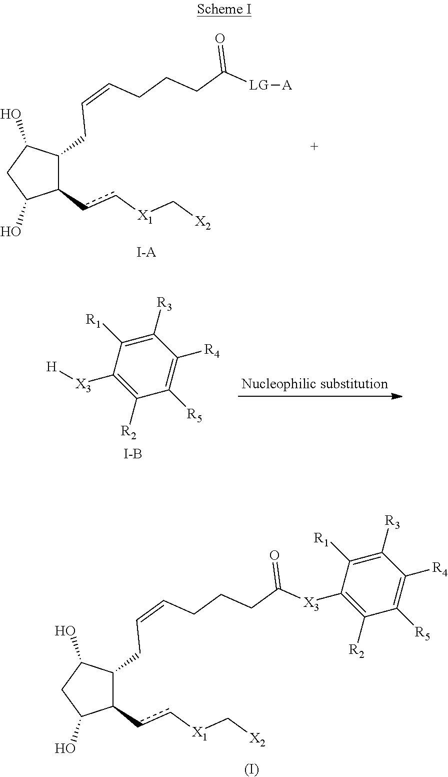

As shown in Scheme I, nucleophile (I-B) reacts with a compound of formula (I-A) containing a carbonyl group and a leaving group attaching to the carbonyl group to form a compound of formula (I). In some embodiment, the compound of formula (I-A) is activated before the reaction. The activation is well known in the art, e.g., by using a coupling agent to transfer it to a more active compound. The reaction is typically carried out in an aprotic solvent (e.g., DCM, NMP, DMF), in the presence of an organic base, such as an aliphatic amine, (e.g., triethylamine, DMAP, and DIPEA) and a coupling agent such as EDC, TBTU, TCTU, HATU, COMU, T3P, and the like. Preparation methods are well known in the art. See, e.g., Montalbetti, Tetrahedron 61, 10827-52 (2005).

Additional products can be prepared from the reaction between compounds IA and I-B include the compounds of the following formula:

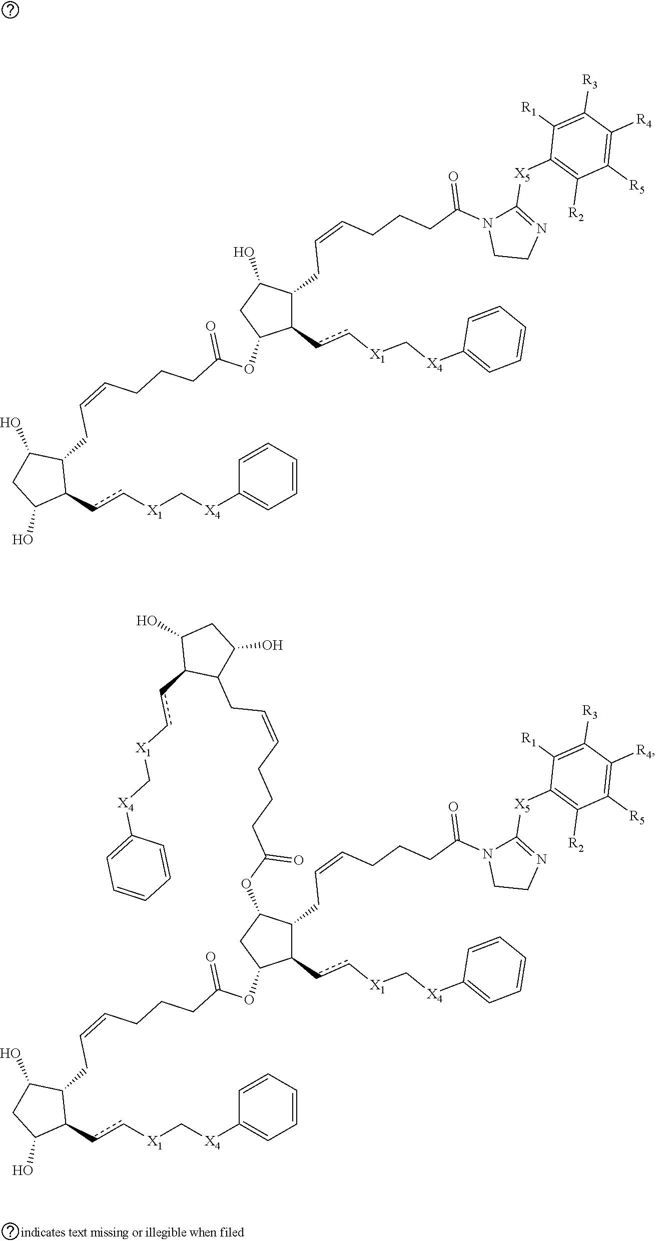

In the formulas above, each of X₄ and X₅ can be X₃, or the groups as defined above.

The compounds of these formulas are also within the scope of this invention.

Both compounds of formula (I-A) and formula (I-B) can be commercially available compounds or known compounds the preparation of which can be readily achieved following known methods.

Exemplary compounds of formula (I-A) include latanoprost acid, travoprost, tafluprost, bimatoprost, unoprost, and the like. Their structures are provided below.

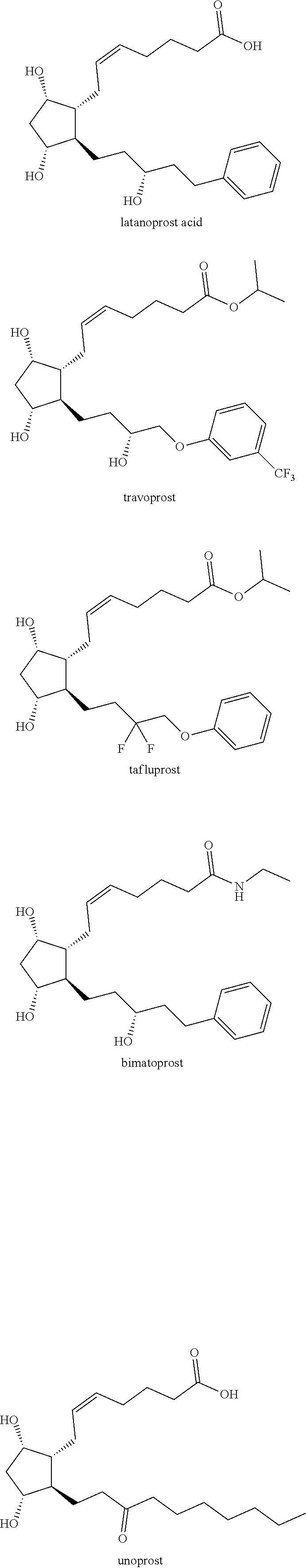 [SMILES: O=C(O)CCC/C=C\C[C@@H]1[C@@H](CC[C@@H](O)CCc2ccccc2)[C@H](O)C[C@@H]1O ; CC(C)OC(=O)CCC/C=C\C[C@@H]1[C@@H](CC[C@@H](O)COc2cccc(C(F)(F)F)c2)[C@H](O)C[C@@H]1O ; CC(C)OC(=O)CCC/C=C\C[C@@H]1[C@@H](CCC(F)(F)COc2ccccc2)[C@H](O)C[C@@H]1O ; CCNC(=O)CCC/C=C\C[C@@H]1[C@@H](CC[C@@H](O)CCc2ccccc2)[C@H](O)C[C@@H]1O ; CCCCCCCC(=O)CC[C@@H]1[C@@H](C/C=C\CCCC(=O)O)[C@@H](O)C[C@H]1O]

Additional suitable compounds of formula (I-A) are those described in publications such as U.S. Pat. No. 6,458,836 B1, US 20200179401 A1, WO2008146105A1, WO 2001068072 A2, and WO 2005068421 A1.

Compounds of compounds of formula (I-B) can be selected from alpha-2 adrenergic agonists such as brimonidine, apraclonidine, guanabenz, methyldopa, and lofexidine with the structures shown below.

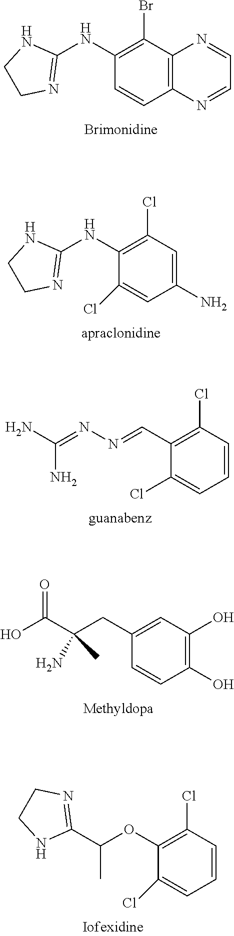 [SMILES: Brc1c(NC2=NCCN2)ccc2nccnc12 ; Nc1cc(Cl)c(NC2=NCCN2)c(Cl)c1 ; NC(N)=N/N=C/c1c(Cl)cccc1Cl ; C[C@](N)(Cc1ccc(O)c(O)c1)C(=O)O ; CC(Oc1c(Cl)cccc1Cl)C1=NCCN1]

More suitable compounds of formula (I-B) include those known as alpha-2 adrenergic receptor agonists. Examples are shown in U.S. Pat. No. 9,089,562 B2, U.S. Pat. No. 9,095,506 B2, U.S. Pat. No. 9,487,527 B2, and U.S. Pat. No. 10,940,205 B2.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

### EXAMPLES

The following non-limiting examples are provided to illustrate particular embodiments. The embodiments throughout may be supplemented with one or more details from one or more examples below, and/or one or more elements from an embodiment may be substituted with one or more details from one or more examples below.

### Example 1: Coupling of isopropyl-7-[3,5-dihydroxy-2-(3-hydroxy-5-phenyl pentyl)-cyclopentyl]-5-heptenoate (latanoprost, LP) with 5-bromo-N-(2-imidazolinyl)-quinoxalin-6-amine; 2,3-dihydroxybutanedioic acid (brimonidine, BM)

### Compound 1 Latanoprost-Brimonidine (“LBJ”) Synthesis

1-Ethyl-3-(3-dimethylaminopropyl) carbodiimide hydrochloride slat (EDC-HCl, 52.47 mg, 2 eq), Brimonidine (40.22 mg, 1 eq) and 4-Dimethylaminopyridine (DMAP, 16.71 mg, 1 eq) were added into a Latanoprost acid solution (80.58 mg, 1.5 eq) in DMSO (10.56 ml). The reaction mixture was stirred at room for overnight. After the reaction, DMSO was removed by lyophilization, and the resulting mass was extracted by brine and DCM. The organic solution was dried on anhydrous MgSO4, filtered, and purified by column (EA/ACN/MeOH=1:1:0.1). The final product was 31.92 mg. FIG. 1 shows the ¹HNMR of Compound 1.

### Examples 2-25: Synthesis of Compounds 2-25

Compound 2-25 are prepared following a procedure similar to Example 1 using commercially available agents.

### Example 26: Hydrolysis Test

### Hydrolysis of Latanoprost-Brimonidine

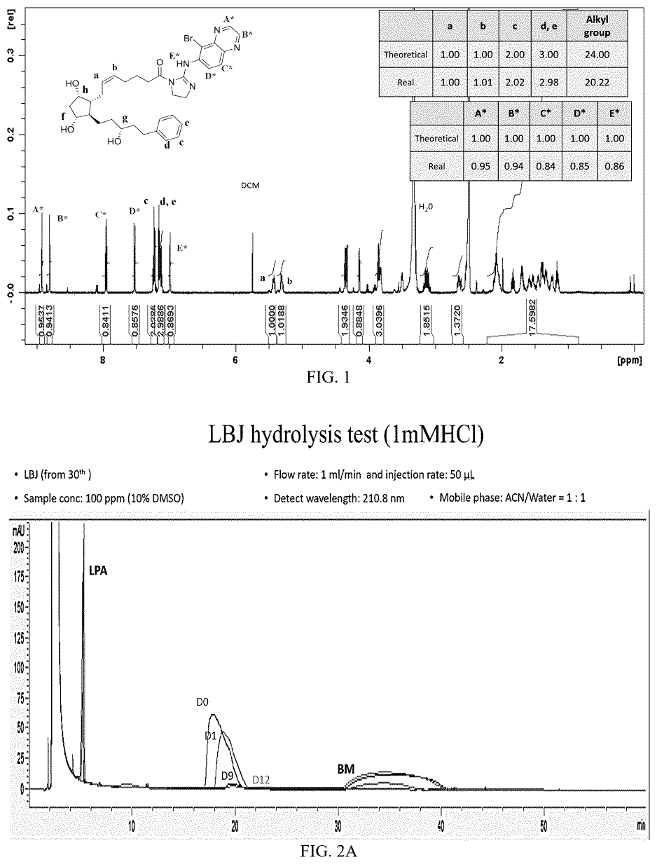

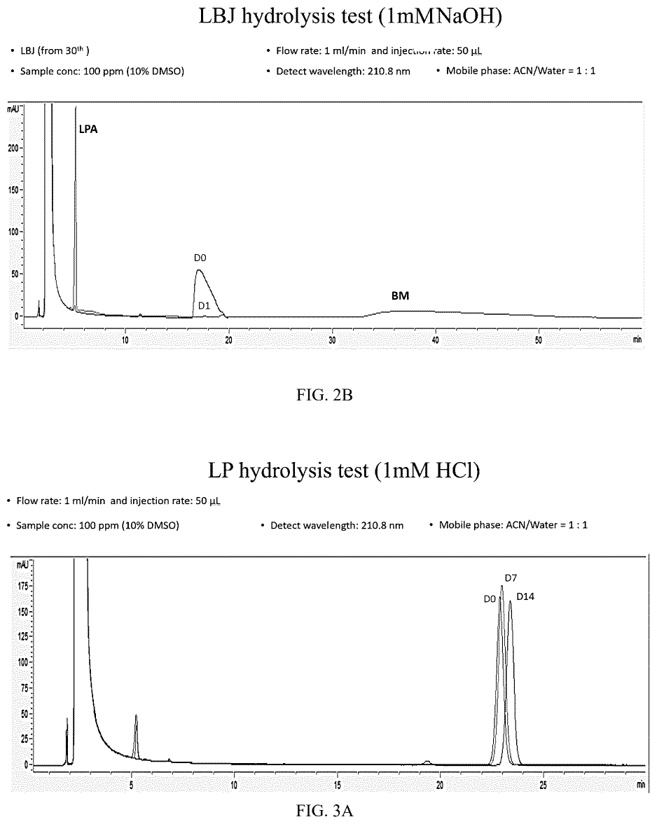

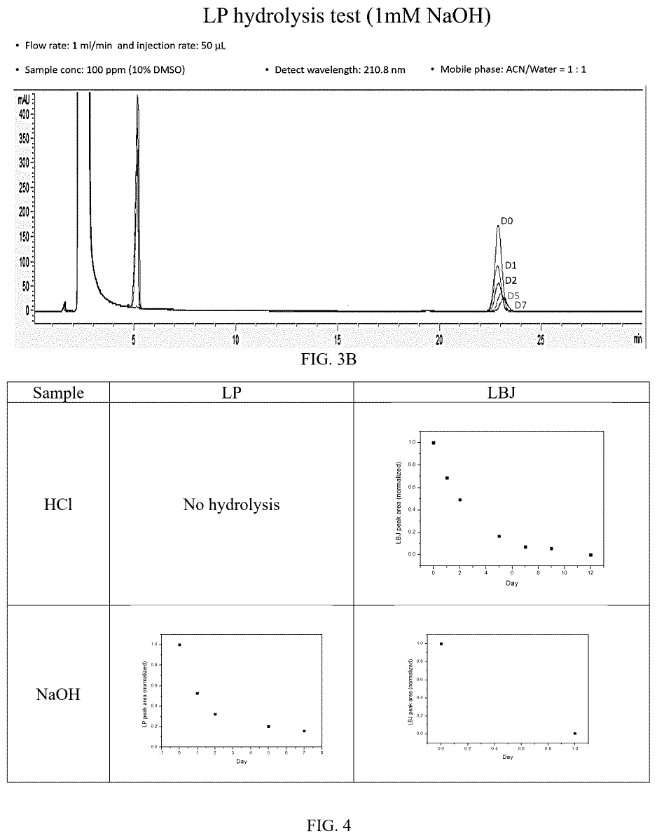

Latanoprost-brimonidine (100 ppm; LBJ, i.e., Compound 1) and latanoprost (100 ppm; LP) in water (10% DMSO) were incubated separately with (1) NaOH (1 mM) and (2) HCl (1 mM) in a 37° C. water bath for up to 2 weeks. Samples (50 μl) removed at different time points were traced by HPLC under the following conditions: the HPLC used was Agilent® 1100 series, the column used was SCpack® ODS-P, the mobile phase used was water/Acetonitrile (1:1, v/v). The hydrolysis results are shown in FIG. 2A (HCl) and FIG. 2B (NaOH) for LBJ and FIG. 3A (HCl) and 3B (NaOH) for LP.

As shown in FIGS. 2A and 4, LBJ was completely hydrolyzed by HCl over a period of 12 days. By contrast, FIGS. 2B and 4 show complete hydrolysis of LBJ by NaOH within 1 day.

In both conditions, LBJ was hydrolyzed into its component parts, i.e., latanoprost and brimonidine, the active pharmaceutical ingredients for lowering IOP.

Latanoprost was not sensitive to hydrolysis by HCl (FIGS. 3A and 4) for at least 14 days, while it was significantly hydrolyzed by NaOH over 7 days (FIGS. 3B and 4).

### Example 27: Effect of Compound 1 on Intraocular Pressure (IOP)

The effect of daily administration of benchmark drugs and Compound 1 formulated for topical instillation was tested in 5 Dutch Belted Rabbits (10 eyes). The following ophthalmic formulations were tested by applying daily drops (approximately 25 μL per drop) to each eye over a period up to 17 days and IOP was measured by standard procedures.

TABLE 1
Formulations for in vivo testing

| Treatment | Concentration (w/v) | Dosing |
|---|---|---|
| Brimonidine tartrate | 0.2% | OD |
| Xalatan ® (latanoprost) | 0.005% | OD |
| Compound 1 | 0.005% | OD days 0-10 |
| | | BID days 10-17 |
| Compound 1 | 0.005% | OD |
| brimonidine | 0.00003% | |
| Brimonidine | 0.2% | brimonidine BID |
| latanoprost | 0.005% | latanoprost OD |

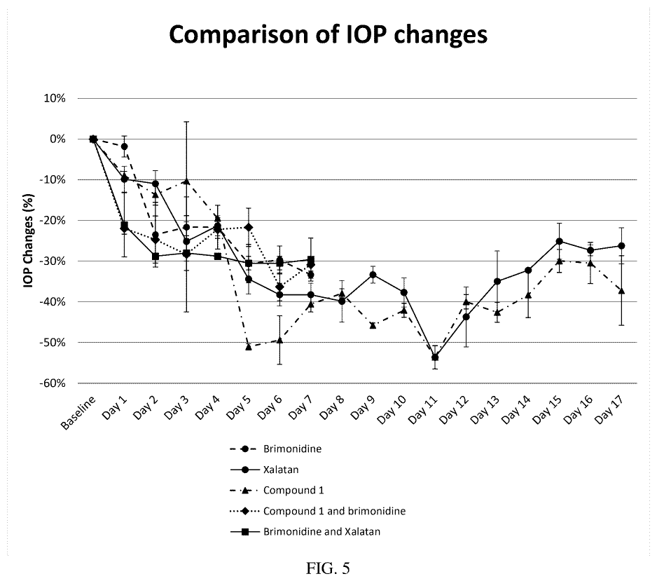

The results are shown in FIG. 5. Administration of Compound 1 daily resulted in a reduction in IOP greater than 50% by day 5. Surprisingly, Compound 1 administered together with a minimal amount of brimonidine (0.00003%) caused a reduction of 22% in IOP after only one day of administration, as compared to a 9% IOP reduction by Compound 1 alone after one day. Brimonidine alone (at 0.2%) only reduced IOP by 2% after one day.

The effect of daily administration of Compound 1 was observed for up to 17 days and showed no less than a 30% lowering of IOP, noting that the dose was increased to twice per day after day 10.

### Example 28: Effect of Weekly Administration of Compound 1 on IOP

Compound 1 was tested to optimize its IOP-lowering effect using other than daily dosing schedule. An ophthalmic preparation was used that contained 0.012% (w/v) Compound 1 and 0.04% (w/v) brimonidine. The preparation was administered as eye drops to Dutch Belted rabbits as described above in Example 27. Administration was twice per day separated by 8 hours on Day 0, Day 7, and Day 14. IOP was measured as previously. The results are shown in Table 2 below.

TABLE 2
Prolonged lowering of IOP by Compound 1

| | IOP | | IOP % |
|---|---|---|---|
| Time point | (mm Hg) | SD | reduction |
| D0 Pre-dose | 16 | 0.55 | 0 |
| D0 Post-dose 8 h | 14 | 0.82 | 13 |
| D1 | 13 | 0.85 | 19 |
| D2 | 11 | 0.59 | 31 |
| D3 | 11 | 0.87 | 31 |
| D4 | 12 | 1.20 | 25 |
| D5 | 12 | 1.36 | 25 |
| D6 | 13 | 0.56 | 19 |
| Post-dose D7 | 14 | 0.82 | 13 |
| Post-dose D7—8 h | 13 | 1.02 | 19 |
| Post-dose D14 | 14 | 0.80 | 13 |
| Post-dose D14—8 h | 12 | 0.66 | 25 |
| D15—AM | 11 | 0.78 | 31 |
| D15—noon | 12 | 0.53 | 25 |
| D15—PM | 11 | 0.90 | 31 |
| D16—AM | 11 | 0.45 | 31 |
| D16—PM | 10 | 0.86 | 38 |
| D17 | 10 | 0.17 | 38 |
| D18 | 11 | 0.18 | 31 |
| D19 | 12 | 0.61 | 25 |
| D21 | 14 | 0.70 | 13 |

Administration of Compound 1 twice on Day 0 resulted in a reduction in IOP that peaked about 3 days later and returned to the initial reduced level by 7 days. A similar pattern of IOP reduction was seen after administering Compound 1 on Day 7 and on Day 14. The results show that an optimized preparation of Compound 1 can maintain lower IOP with a dosing schedule that can be more easily complied with by a patient, as compared to, for example, a daily dosing schedule.

### Other Embodiments

All of the features disclosed in this Specification may be combined in any combination. Each feature disclosed in this Specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I), 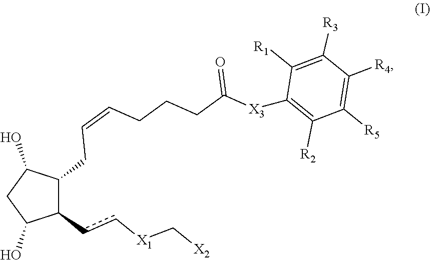
in which
each of R₁, R₂, and R₅, independently, is H, halo, or OH;
each of R₃ and R₄ is H, OH, or amino, or R₃ and R₄, together with the phenyl ring they are bonded to, form a quinoxaline ring;
X₁ is CO, CHOH, or CF₂;
X₂ is C₄-C₆ alkyl, C₄-C₆ alkyl-O—, aryl-O—, or aryl-CH₂—;
X₃ is 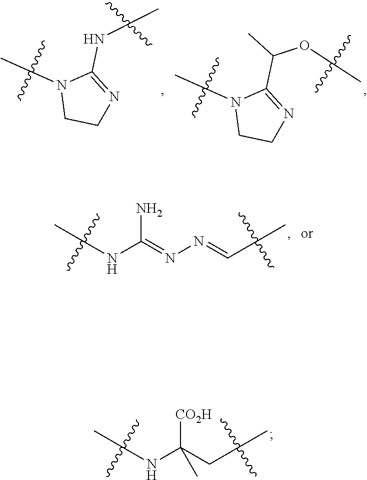
and [symbol]
is a single or double bond.

2. The compound of claim 1, wherein R₁ is H, Cl, or Br.

3. The compound of claim 1, wherein R₂ is H or Cl.

4. The compound of claim 1, wherein R₅ is H or OH.

5. The compound of claim 1, wherein each of R₃ and R₄, independently, is H, OH, or NH₂.

6. The compound of claim 1, wherein R₃ and R₄, together with the phenyl ring they are bonded to, form a quinoxaline ring, and the quinoxaline ring is unsubstituted or substituted with amino, hydroxyl, or halo.

7. The compound of claim 1, wherein X₁ is chiral CHOH.

8. The compound of claim 1, wherein X₂ is 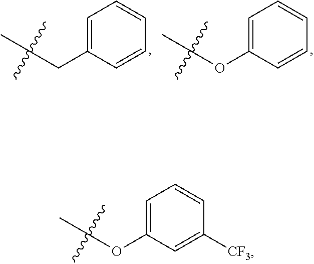
or n-hexyl.

9. The compound of claim 1, wherein X₃ is

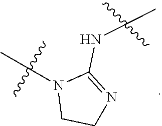 [SMILES: *NC1=NCCN1*]

10. The compound of claim 1, wherein [symbol] is 1 double bond.

11. The compound of claim 1, wherein the compound is a compound of formula (II): 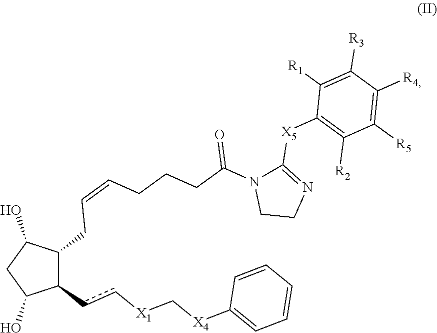
in which
each of R₁, R₂, and R₅, independently, is H, F, Cl, or Br;
each of R₃ and R₄ is H or NH₂, or R₃ and R₄, together with the phenyl ring they are bonded to, form a quinoxaline ring;
X₁ is CHOH or CF₂;
X₄ is CH₂ or O; and
X₅ is NH or CH(CH₃)O.

12. A method of preparing the compound of claim 1, the method comprising the step of reacting a compound of formula (I-A) and a compound of formula (I-B): 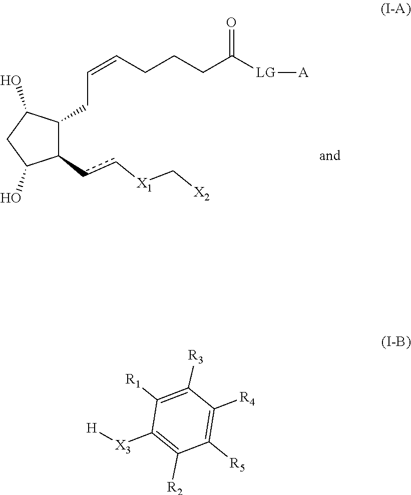
in which LG-A is a leaving group and X₃H is a nucleophile.

13. The method of claim 12, wherein LG-A is halo, 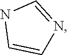
anhydride, N₃, or OH; and X₃H is OH or amine.

14. A pharmaceutical composition comprising the compound of claim 1 and a pharmaceutically acceptable salt.

15. The pharmaceutical composition of claim 14, further comprising brimonidine.

16. A method for treating glaucoma comprising the step of administering the compound of claim 1 to a subject in need thereof.

17. The method of claim 16, wherein the administering step is carried out daily.

18. The method of claim 16, wherein the administering step is carried out once per week.

19. The method of claim 18, wherein the administering step comprises applying the compound or pharmaceutical composition twice.
